(19) United States
(12) Patent Application Publication
(10) Pub. No.: US 2009/0112293 A1
(43) Pub. Date: Apr. 30, 2009

(54) Device for Carrying Out a Treatment

(76) Inventor: Paul Haslauer, Salzburg (AT)

Correspondence Address: NIXON & VANDERHYE, PC, 901 NORTH GLEBE ROAD, 11TH FLOOR, ARLINGTON, VA, 22203, US

(21) Appl. No.: 12/084,623

(22) PCT Filed: Dec. 7, 2006
(86) PCT No.: PCT/EP2006/011777

§ 371 Date: Dec. 22, 2008

(30) Foreign Application Priority Data
Dec. 9, 2005 (DE) 10 2005 059 389.5

(51) Int. Cl. A61H 33/00; A61F 7/00

(52) U.S. Cl. 607/85; 4/548

(57) ABSTRACT

An improved device for carrying out a treatment comprising the following features: the treatment is carried out with the film hanging only slightly down, and, in order to carry out the treatment, the film can brought into a position in which the lateral portions of the film, which lead to its lateral edge support, ascend in a relation to a horizontal plane at an angle smaller than 45°, the film being of such a type of such a configuration that its top face is slidable at least with additional use of treatment medium and/or lubricant.

The invention relates to a device for carrying out a treatment according to the preamble of claim 1.

Heat treatments, for example in the form of packs, bath treatments or else in the form of steam bath or herbal steam bath treatments, have always been extremely popular. Treatments of this type not only serve to improve general well-being but can also help to stabilize and improve health.

Two aspects must, for example, be considered in heat treatments of this type.

On the one hand, bath treatments, especially in spas, health clubs, hotels and the like, are known to be by no means inexpensive. The reason for this is that the bathing media which are used must, simply for reasons of hygiene, be removed once a treatment has been carried out and be replaced by hygienically optimum bathing media for carrying out a subsequent treatment. If particularly effective natural substances or other additives are additionally used, this generally also requires considerable amounts which greatly increase still further the costs of such a treatment.

On the other hand, however, it must also be borne in mind that, owing to the markedly increased life expectancy of broad sections of the population, the cardiovascular system must be treated with due care when carrying out heat treatments, especially in the case of elderly persons. Treatments which are carried out over a large surface area, and thus cause excessive heating, can be problematic.

In order to reduce the amount of bathing media required, for example the amount of peloid or of bath additives, DE-PS 30 46 628 has already proposed an improved device for the administering of peloid full-length or sitz baths requiring for each individual bath therapy only a relatively small portion of the bathing medium which could previously be used only once for hygiene reasons. This was achieved by providing in the tub an insert which is configured as a liquid-tight foil for separating the peloid bath liquid which is to be introduced above and below the foil. When carrying out a bath treatment, someone can thus lie down in the space above the liquid-separating foil, so only the bath liquid which enters into contact with him must be exchanged for hygiene reasons. This allows the volume of bath liquid and bath additives, peloid, etc. to be considerably reduced compared to conventional treatments. However, the problem of possible excessive heating of the body remains.

EP 0 144 571 B1 has also disclosed a device for the administering of packs, in which the packs are positioned when carrying out a treatment only at specific positions to be heated strongly, wherein the use of insulating mats, above all in the leg and neck regions, produces the advantage of allowing, especially in the case of elderly persons, excessive heating of a heat and press-on medium acting below a contact and press-on foil to be avoided.

Both of the above-mentioned previously known tub arrangements therefore use a respective foil which is fixedly clamped and held to the peripheral edge of the tub. In the case of the foil tub according to EP 0 144 571 B1, the additional use of a single-use foil is further proposed in the administration of packs for hygiene reasons. Thus, in the case of the tub known from the above-mentioned publication, insulating materials can be placed onto the press-on foil initially at the points at which excessive heating is to be avoided (for example in the form of towels or hand towels in the throat, neck or head region) in order then finally to place thereon the single-use foil which therefore comes to rest in certain portions on the press-on foil and in certain portions on the previously applied insulating materials. A person to be treated, to the surface of whose body the respectively desired packs or pulpy or lubricating media can be applied, then lies down on this arrangement. After the treatment, the single-use foil is discarded.

A device using a foil has become known, for example, from DE 88 09 687 U1. This is a plastics material insert for a sitz bath tub in the form of a foil which is inserted with a precise fit into the sitz bath tub and can easily be removed after use thereof without losing much time as a result of complex cleaning or disinfecting operations.

The object of the present invention, on the other hand, is to provide a new device which opens up new possibilities in the administration of a treatment.

According to the invention, the object is achieved in accordance with the features specified in claim 1. Advantageous configurations of the invention are specified in the sub-claims.

The present invention has designed an entirely new device which can be used to carry out an entirely novel treatment.

That is to say, the device according to the invention is distinguished by the fact that it has a contact surface which preferably consists of a foil and sags downward only comparatively little even when carrying out the treatment, i.e. when a person is located on the foil. This produces a somewhat bowl, plate or wok-like contact surface even if the foil is held at least in certain portions on a tub-like carrying device, namely at a peripheral edge of the tub.

In other words, the contact surface or foil should ascend, even in the loaded state when carrying out heat treatment, for example extending laterally away from the body, only at a comparatively small angle in relation to the horizontal, which angle may be no greater than 45° and preferably smaller than 40°, 35°, 30° or even smaller than 25°. This produces a contact surface which is deflected in a comparatively only slightly bowl-like or concave manner and the outer holding edge of which, at which the foil is carried and supported, is at the same level as the person located on the foil. In other words, the edge of the holding foil can be located just above the person located on the foil or else at the same level as the person lying on the foil, for example at the level of the head, chest or stomach region. Even if the foil is supported at a different point on the outer edge, there is provided at least one diversion point which is arranged relative to the person lying on the foil at a level such that the foil ascends only comparatively slowly and slightly at this diversion point away from the body, i.e. from the point where the foil sags most markedly owing to the loading, in order to ensure a contact surface in which the depressions are configured so as to be just comparatively shallow and in which the relevant person can easily slip and slide.

The invention allows a pleasant treatment, which involves a certain degree of pampering and/or massaging, to be carried out. In the past, such treatment was possible only passively as a result of a second person, for example a masseur or a therapist, carrying out a massage on the body of a guest or patient.

In contrast thereto, the device allows the bath guest himself to carry out and control the treatment as a result of body movements initiated by the guest himself and owing to the sliding and frictional effect thereby initiated.

However, even if some bath guests do not wish themselves to be responsible for carrying out the treatment in a manner substantially controlled by them, but rather prefer a certain degree of passivity in this regard too, the invention offers additional possibilities. Thus, for example, the foil carrying the guest, which will be referred to hereinafter in some cases also as the sliding foil, can also be withdrawn laterally or on the foot side and returned to the same position. This can be ensured, for example, by one or more movable support or suspension means, for example in the form of lateral rolls, via which the foil can, for example, be moved back and forth from left to right and vice versa. Alternatively or additionally, it is also possible to provide additional influencing means below the foil in order to move the position of the foil and thus the guest located thereon. It is also possible to change the center of gravity of the carrying or tub means, for example by raising, lowering, tilting about a tilt and/or pivot axis extending in the longitudinal direction of the carrying and tub means or in the transverse direction and/or rotating about a more vertical axis.

A configuration of this type now opens up the possibility, using addable media which are free-flowing, paste-like, pulpy or creamy, of even the slightest body movement causing a person located on this foil to slip and slide on this foil. As a result, the foil carries out rubbing and sliding movements at the back, shoulder and buttocks regions. In other words, the invention provides a device in the form of a cushion, couch, etc. with the aim of promoting and/or allowing sliding contact using a lubricant.

Raisable and/or lowerable carrying and/or support means which are provided below the foil and act on the underside of the foil, for example in the form of padded and/or inflatable means, are also conceivable. Free-flowing or gaseous medium such as air can for example also be blown or pumped into a closed foil space, thus allowing the generation of influences which exert differing compressive loads and generate and/or intensify the desired effects when carrying out the treatment.

What is known as the “wok effect” is important within the scope of the invention. Owing to the specific bowl or plate-like wok cross-sectional configuration, this is known to cause treatment materials and substances applied to the foil, which substances which may be free-flowing, creamy, paste-like, etc., to flow or slide, following gravity, above all to the lowest point at the center of the foil. The climbing-in of the guest presses the sliding foil downward. This gives rise on all sides to an oblique position of the foil. As a result of the sliding movements of the guest and as a result of his body weight, the bulk of the lubricant, which was previously applied to the guest's body and/or to the surface of the foil, is pressed out laterally between body and foil. The wok effect forces this portion to move at all times in contact between body and foil. The guest can in this case pick up, as it were, “excess” lubricant and purposefully supply it for rubbing into his body. This further reduces the amount of single-use material applied.

In order to improve the operativeness of the foil and the prolonged use thereof, the foil can also have two or more layers.

Generally speaking, in order to be able to accommodate body weights, the foil should comprise a sufficiently strong and durable layer, for example be provided with a fabric reinforcement, so the foil can in any case accommodate with sufficient security the carrying forces of a person. A foil thus formed can on its upper side be provided, laminated or generally equipped with a layer having optimum sliding properties. A cavity, which is also filled with a free-flowing, gaseous or gel-like material, can also be formed between the top sliding layer and the carrying layer located therebelow, thus allowing the top sliding layer to be leveled. Even if the carrying layer sags, it can thus be ensured that the upper sliding foil comes to lie substantially horizontally.

A further, more sagging foil can however also be provided below the carrying foil, thus preferably likewise forming again a closed chamber system. If at least one supply opening leads into and optionally a further supplementary discharge opening (although this is not necessary) leads out of this chamber, the lower cavity can for example be filled with circulating hot air as a heat medium. This bottom foil is preferably also fabric-reinforced in order to act as a safeguard, should the upper carrying foil unexpectedly tear. In any case, this lower chamber system forms a closed heating chamber, thus allowing a tub body to be dispensed with entirely. A device for carrying the foil design is all that is required to allow the desired temperatures to be set when carrying out the treatment, to be maintained over the course of the treatment or else to be changed over the course of the treatment. In other words, even if a person lying on the top sliding foil should press this foil into contact with the preferably fabric-reinforced carrying foil located therebelow, the desired conveyance of heat and the heating are on the whole maintained, because the bottom chamber cannot be compressed. In order to maintain a good filling pressure of the heat medium in the bottom chamber, a preferably settable or adjustable restrictor or an outlet opening having a smaller diameter than the inlet opening can be provided in the discharge.

Finally, this system also offers the advantage that, prior to the treatment, the introduction of warm air causes the top and the second foil to be bulged upward somewhat in a convex manner, thus greatly simplifying cleaning (owing for example to cleaning liquid). If a bath guest lies down on the top foil, the first and second foils are pressed downward accordingly, although not so far as to touch the third and thus bottom foil. This multiple-chamber system therefore performs a dual function, namely for securely carrying and holding a bath guest on the top foil and for providing a heatable space.

Undesirable excessive heating of the head can also be prevented by means of a specific head rest including head and neck pieces.

In addition, there can preferably also be provided on the side of the device grips which can be folded open and/or over or at least range-positionable grips or other holding means which enable the bather or guest to support himself thereon using his hands and possibly also to allow his body, by actuating his arm muscles, to slide back and forth, to push, to rotate or otherwise to turn accordingly in the slight depression in the foil. At the same time, this also prevents undesirable sliding out of the foil rest and facilitates purposeful climbing in and out. If slipping too far into the bowl or wok-like contact surface is to be prevented, an adjustable foot rest can for example also be provided; this increases convenience, especially for relatively short guests.

Finally, there can also be positioned on the foil a few wedges, lateral support wedges, etc. which at least to a certain extent limit and restrict the sliding movement in order if appropriate to prevent a sense of anxiety which some bath guests may experience. These additional means, which may offer at least some support to the body, in the form of wedge-like pads, inflatable bodies, etc. can on the whole be connected to the device carrying the foil, for example using suitable devices, thus allowing them to be swiveled in and out without becoming lost. Further additional handles or hand grip portions, which a bath guest can use to steady himself, can also be configured on these additional positionable wedges or limiting bodies. Finally, the position and posture of the body can also be changed using wedges of this type, for example by means of a wedge-like cushion which can be placed under slightly raised and angled knees, or for example by means of a cushion in the upper shoulder or at the start of the back region, etc.

Further advantages, details and features of the invention will emerge subsequently from the exemplary embodiment illustrated with reference to the drawings, in which specifically:

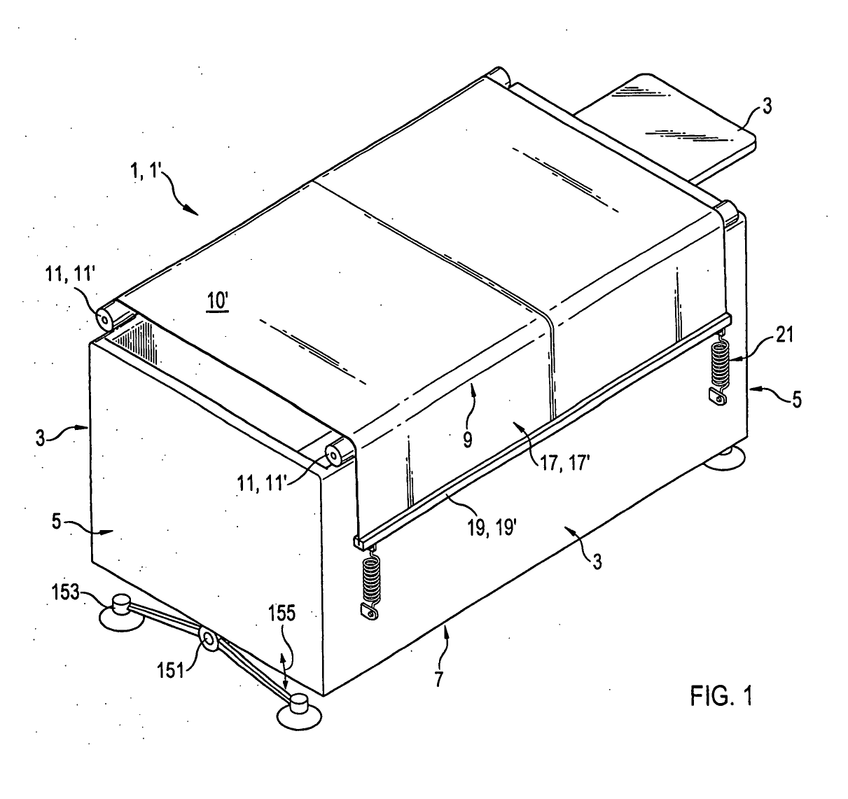

FIG. 1 is a schematic three-dimensional view of a device according to the invention with a contact or sliding foil pulled taut on the upper side;

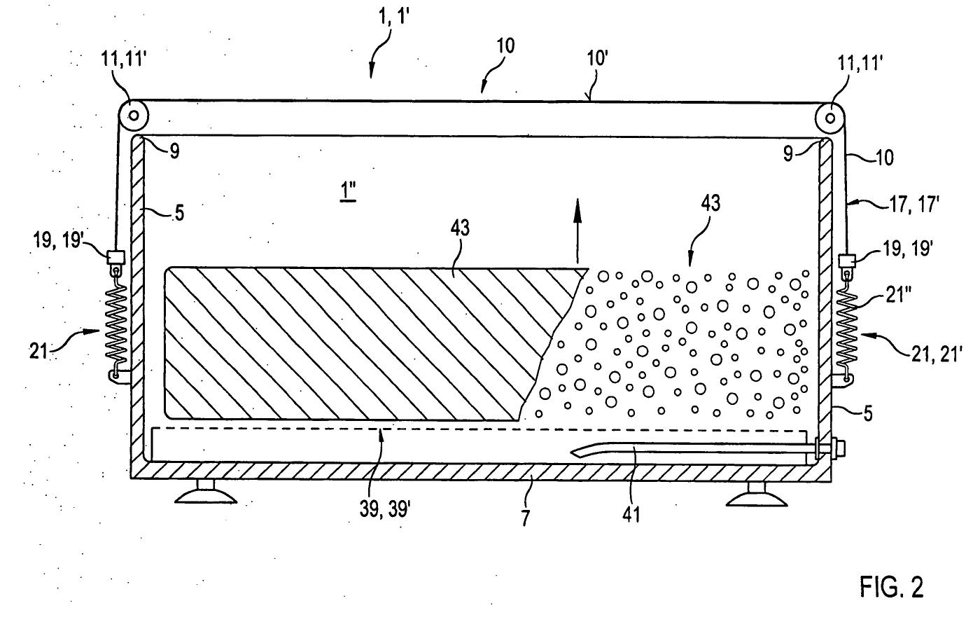

FIG. 2 is a schematic cross-sectional view, running in the vertical direction, transversely to the longitudinal direction of the device;

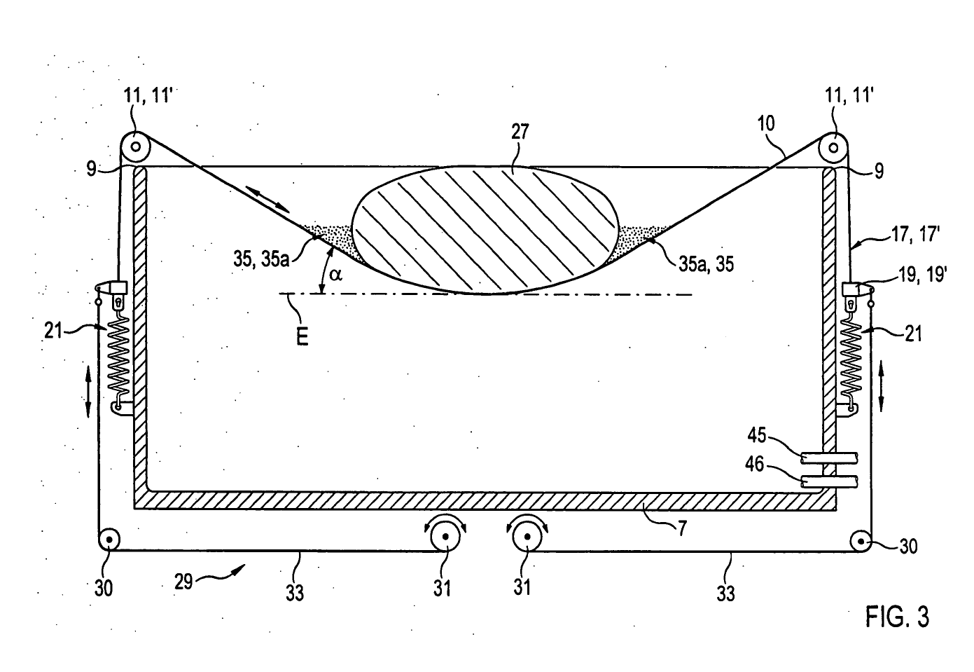

FIG. 3 is a view corresponding to FIG. 2 but with a person lying on the contact or sliding foil;

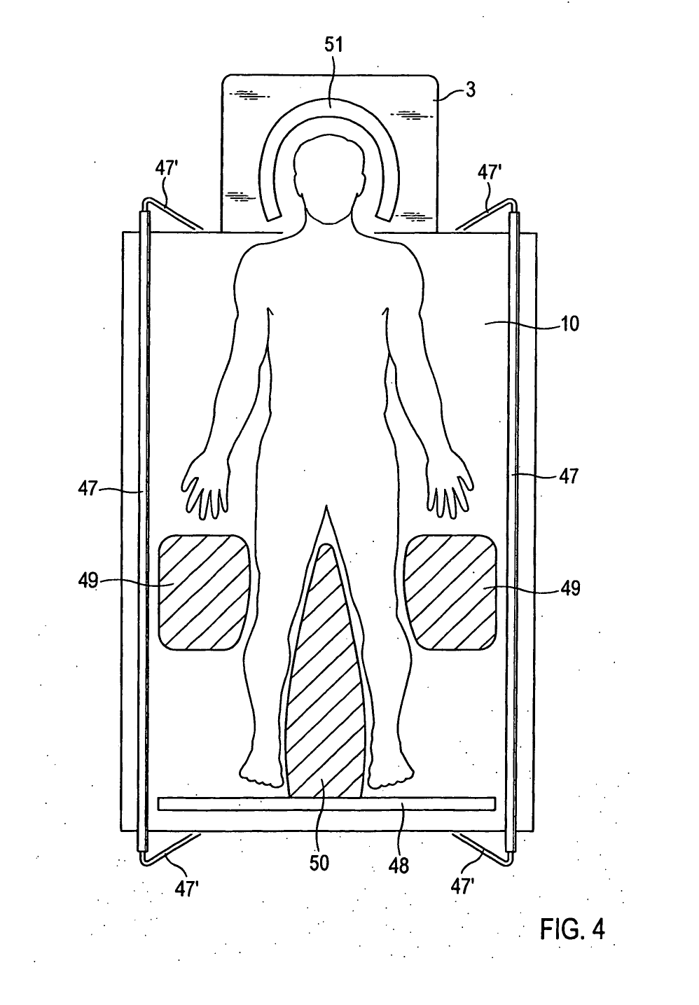

FIG. 4 is a schematic plan view during the carrying-out of a treatment using additional auxiliary means;

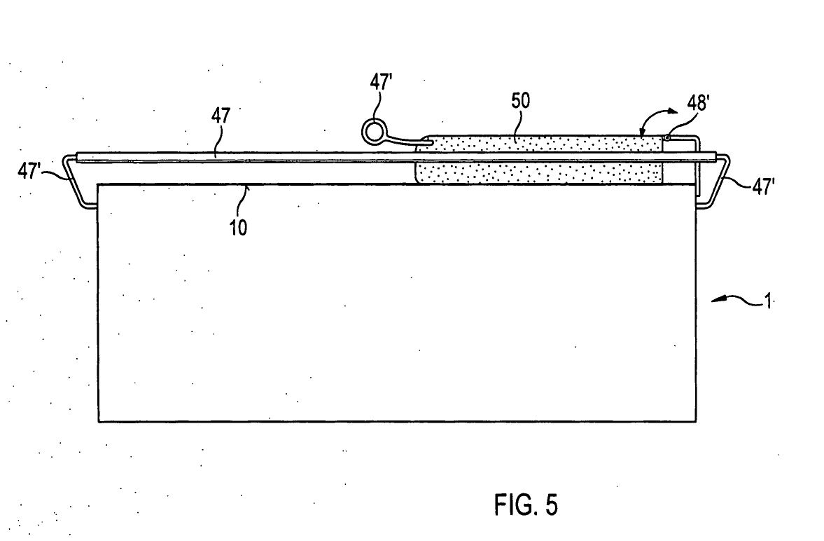

FIG. 5 is a schematic side view of the device showing an inner and/or knee part with gripping means resting thereon;

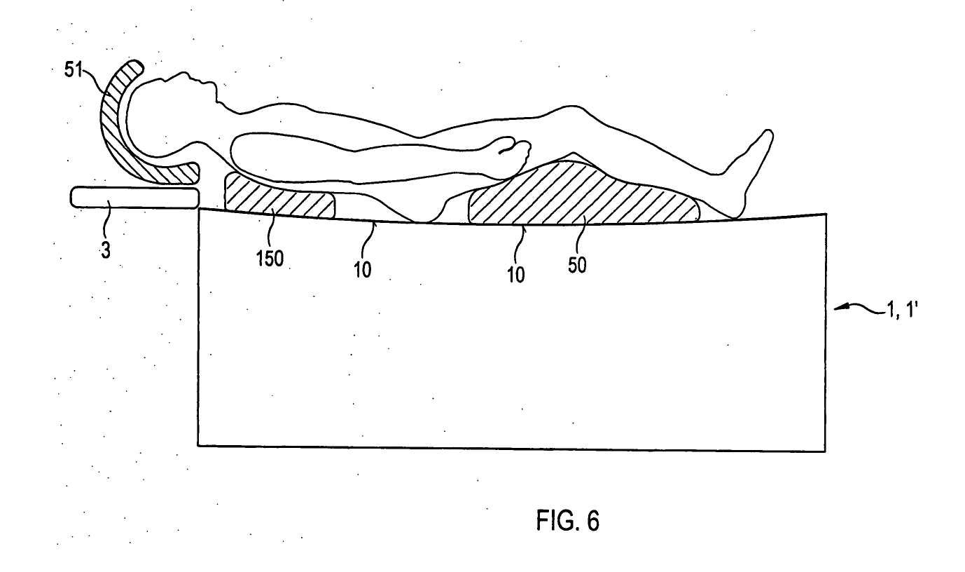

FIG. 6 is a further lateral view indicating a knee part and a back piece part for changing the position in which a bath guest lies; and

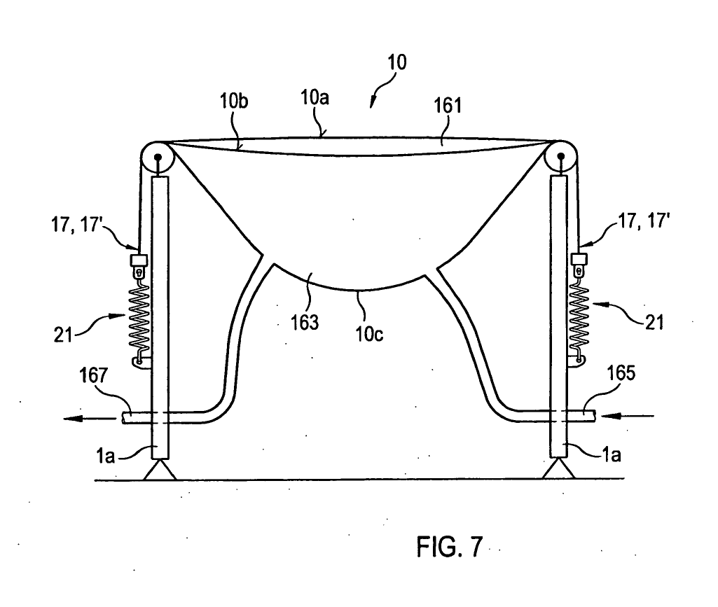

FIG. 7 is a cross-sectional view through a multi-walled sliding foil, forming foil gaps.

FIGS. 1 to 3 show a first exemplary embodiment of the invention.

It comprises a carrying device 1 which in the exemplary embodiment shown consists of a basic tub element 1′ comprising two opposing longitudinal sides 3 and two end faces 5 which are likewise set apart from one another in the longitudinal direction. The carrying device 1 has a base 7 and is open in the upper edge region.

In the exemplary embodiment shown, diversion means 11, which in the exemplary embodiment shown consist of diversion rolls 11′, are provided at the level or at least approximately in the region of the upper peripheral edge 9 on the carrying device in the form of the basic tub element 1′, i.e. in the exemplary embodiment shown at the two longitudinal sides 3. The diversion means 11 can also consist of the upper edge 9 of the carrying means 1. Rotatable rolls are however preferred, as this allows the foil 10 (which will be examined hereinafter in greater detail) to be moved more easily back and forth from left to right and vice versa. The axial mounting of the rolls 11 is not shown in detail in the exemplary embodiment shown. The supporting of these diversion means, preferably in the form of the diversion rolls 11′, can be carried out in the region of the upper edge 9, on the inside or outside 3 of the tub body or else outside the tub body on a stand or base surface 15, and be supported at this location.

The diversion rolls 11′ may in this case preferably be soft or resilient in their configuration in order, in particular, to rule out risks of injury when climbing in and out.

In the exemplary embodiment shown, it may be seen that the aforementioned contact and/or sliding foil 10, which at the edge of the tub is guided out of the region of the tub via the diversion means 11 and guided further downward on the outside of the tub, does not sag in the region of the tub over a relative height or even would not reach close to the base of the tub. On the contrary, the exemplary embodiment has been selected in such a way that the sliding foil 10, at least in its starting position, is held via a securing portion 17, preferably at an external edge region 17′, via a holding or securing means 19, preferably in the form of a rail-like tensioning frame 19′, via an actuating and/or support means 21.

In the exemplary embodiment according to FIG. 1, it is in this case indicated that the support means 21 comprises a tensioning device 21′ which can, for example, comprise a spring means 21′ or a spring force storage means 21″, for example in the form of one or more helical springs, which is held or supported at a suitable point. This ensures in the starting position that the aforementioned contact and/or sliding foil 10 is, when unloaded, almost fully stretched above the interior 1″ of the tub and thus extends with substantially no sagging.

The spring force storage means 21″ is in this case configured in such a way that, when a person lies down on the device, then the contact or sliding foil 10 dips only slightly, in a manner comparable to the cross-sectional view according to FIG. 3, into the interior 1″ of the carrying device, preferably in the form of the basic tub element 1′.

In the exemplary embodiment shown, the spring force storage means 21″ is in this case preferably adapted in such a way that, for an average body weight, the foil ascends laterally from the guest located on the foil or the person 27 carrying out the treatment only comparatively flat in relation to the support and diversion means 11, preferably at an angle α in relation to the horizontal plane E that is smaller than 45°, preferably smaller than 40°, 35° and in particular smaller than preferably 25°, 20°, 15°, 10°, 5°. This therefore forms a contact surface 10′ which is shaped only slightly concavely.

The carrying-out of the treatment will be examined hereinafter in conjunction with further configurations of the device.

In the starting ready position shown in FIGS. 1 and 2, the top sliding foil 10 is therefore more or less planar and flat, i.e. tensioned into its at least approximately horizontal starting position. In this position it is now, for example, easy for a bath attendant, therapist, masseur or other assistant to apply to the foil appropriate media—generally, in other words, free-flowing, pulpy or paste-like, creamy or other media which will also be referred to hereinafter as lubricants for the sake of brevity. Moreover, after carrying out the treatment, it is also easy in this starting position, which may be returned to, for the respective person, for example in the form of the bath attendant, to clean the surface of the foil, as no residues or puddles are left behind.

Before carrying out the treatment, it is first advisable to perform what is known as an initial anointment on a person to be treated, who can carry out the treatment himself or, for example, allow the bath attendant to treat him. Subsequently, the sliding foil 10 is, for example, lightly wetted with a preferably warm medium such as warm water and a thin layer of the lubricant is optionally applied to, sprayed onto, distributed over, rubbed into, etc. the foil.

The bath guest can then, for example, lie face down on the foil. As the lateral edge of the device is slidable and the aforementioned diversion rolls are preferably padded, it is easy for the bath guest conveniently to climb into the device, i.e. to lie down on the upper side of the foil.

If, therefore, a guest lies down on the foil 10, his own body weight will cause the foil 10 to bulge in an at least slightly concave manner counter to the force of the spring tensioning means 21′ and accordingly to dip somewhat into the interior 1″ of the tub. The degree to which the foil 10 penetrates the interior of the carrying device 1, which is configured in the form of a basic tub element 1′, determines the skin surface involved in the sliding. The frictional resistance between the body 27, on the one hand, the lubricant located therebetween and the sliding foil, on the other hand, is likewise determined by the depth of insertion and thus the frictional surface.

Once the bath guest 27 has therefore placed himself on the foil, for example in the face-down position, and his body dips somewhat into the interior 1″ of the tub, the assistant, for example in the form of the bath attendant, can moisten the bath guest's back with warm water and rub in a portion of the lubricant and distribute it over the guest's body through extensive distributing movement.

After the above-mentioned initial anointment of the bath guest, any remaining lubricant has then merely to be applied to or poured onto the bath guest's back, shoulder portion and leg region. The bath guest now turns onto his back. In this case, depending on the consistency of the lubricant 35, the bulk of this lubricant is displaced outward from the space between the body 27 and the sliding foil 10, as is indicated by reference numeral 35a in FIG. 3. If the bath guest moves his body, any remaining lubricant is constantly guided up to the bath guest's body as a result of the oblique position of the sliding foil 10. This displacement of the excess lubricant is a special experience for the bath guest. This pleasantly stimulates the tactile receptors even without any actual active movement.

This experience is entirely comparable with passive immersion. Without having to be specifically instructed to do so, the bath guest rubs the medium over his body using his hands and massages it in. A dry mixture of crystal sugar, healing earths and the like can, for example, then additionally be applied. Until the sugar crystals have dissolved, pleasant peeling on the skin is possible. Subsequently, the above-mentioned additionally added substances such as crystal sugar, healing earth, etc. mix with the originally applied lubricant. This peeling, the duration of which is limited, produces a very pleasant feeling and is good for the skin. This also leads to a rise in, for example, the salt concentration throughout the lubricant. This rise in the salt concentration is desirable as what is known as the retard effect. The addition of tubular rolls right at the outset or at a later point in time can bring about what is known as a ball bearing effect. This causes a marked increase in the sliding effect. Macadamia nut oil is said to have such properties. On the other hand, added granules (soluble or insoluble grains) can increase resistance and facilitate or intensify the peeling effect.

For the treatment, an appropriate ambient climate is preferred. A sliding bath in a space with currently conventional air exchange would only lead to undesirable drafts and possibly to drying-out of the lubricant. Therefore, the described device is preferably used in a steam bath space, for example with rising temperature and rising atmospheric humidity. In the case of a two-person arrangement, the use of a climatic steam bath tent as an outer frame, which can be used when opened as a resting place and when closed as a herbal steam bath environment, is conceivable.

If a single device is to be used in a generally air-conditioned space, non-contact covering with a heat-reflecting foil (for example with an aluminum coating), which can be set apart from the body using a device, is advisable to avoid the feeling of drafts, cold and drying-out of the medium.

Once the treatment has been completed, before the bath guest leaves the device, a fan or a comparable means arranged on a cabin ceiling can be used to inject water which is distributed by the centrifugal force throughout the space and also sprayed down in the region of the device and thus onto the bath guest.

If a system for producing and/or distributing small sheets of ice is provided in the respective space, these sheets of ice could be scattered into the space by a sheet distributor connected to a fan. The guest will accordingly enjoy refreshment and cooling of his body, the likes of which he has scarcely ever experienced, after an intensively heating sliding bath. The water distributed by the fan on the cabin ceiling additionally cleans the bath guest and the lying surface.

Merely for the sake of completeness, it should be noted that for climbing in and out of the device, a lifting and lowering means which is located below the foil 10 can also be provided below the foil in the interior of the device or basic tub element.

A lifting and lowering means of this type is however not shown in detail. Reference is therefore made to known solutions.

Nevertheless, a further modification and addition will be shown hereinafter, still with reference to FIG. 2.

In the exemplary embodiment according to FIG. 2, a spacer means 39 which rests on the base of the tub or is otherwise supported is provided in the interior 1″ of the tub, for example in the form of a perforated metal spacer sheet 39′. Located therebelow is a heating means 41. On the introduction of water or another medium, not air but rather this free-flowing medium is heated, because the heat also reaches over through the openings in the spacer means 39 into the space located above the spacer means 39 and spreads at this location.

Above the spacer means 39 it is shown in the exemplary embodiment that that there may be provided at this location not only stationary but rather also movable floating bodies, for example in the form of inflatable inserts 43, which are located in this space, are inserted here and/or can be folded or raised up to the sliding surface, i.e. the sliding foil 10, from below. In the event of uncontrolled movement of a bath guest above the sliding foil, they can counteract an uncontrolled movement process. This can also prevent states of anxiety which certain bath guests may experience. The body thus senses a certain counterpressure below the foil, for example as a result of the use of a foam-like insert, a padded insert, etc. Floating and/or inflatable bodies, mattress-like inflatable bodies, etc. provided in this space below the foil can also provide support in the region of the back, buttocks and/or knee. In FIG. 2, the inserts 43 are shown at the bottom resting on the perforated metal spacer sheet 39. If, however, accordingly sufficient water, for example water heated accordingly as desired, were then introduced into the tub (through a feed not shown in detail in FIG. 2), the insert or inserts 43 would also rise in tandem with the water level until they rest against the underside of the foil 10 in each case with the desired contact pressure.

The right-hand portion of FIG. 2 merely indicates that there can also be provided, instead of one or more floating bodies which can, for example, be inflated in a foam rubber-like or mattress-like manner, below the foil a buoyancy means, for example in the form of a floatable granular inlay 43 which comprises a large number of floating grains and is able to float when water has been added, the grains then acting on the foil from the underside in accordance with the buoyancy forces generated by the granular material. Any desired further modifications are at all times possible.

The cross-sectional view according to FIG. 3 shows as an alternative thereto that there can be associated with the device, i.e. the interior of the tub, at least one supply tube 45 and an outlet tube 46 via which, for example, a free-flowing medium, for example air, can flow into the interior of the tub and flow out again through the outlet tube 46. This allows, for example, steam which is warmed or heated by an external heater or, for example, using a steam heater also to be supplied to the interior. Constant circulation allows the temperature to be deliberately maintained and/or changed at this location. Equally, liquid, and not only gaseous, medium can also be supplied at this location.

It has already been pointed out that a guest who initially lies down on the sliding foil is partly responsible, owing to his inherent weight, for the sliding foil 10 to a certain extent sagging in a concave manner. The extent of the sagging can however be limited by various measures. Limitation of the sagging of the sliding foil 10 and of the immersion into the device or into what is known as the basic tub element from below can also be influenced and controlled by differing forces and measures counteracting the immersion, including by the foil which is used or the materials which are used for the foil itself, which may allow a differing degrees of bulging and sagging, because for example a resilient or at least slightly resilient material is used for the foil. Although all of these measures determine and/or alter the depth of insertion, “unsoft” mounting is in this case imparted. Therefore, by means of an adjustable tub base which is located in the tub, a foam rest or a floating body inlay can be provided in the tub so as to float in warm water in order to allow corresponding buoyancy forces from the underside of the foil to act on the foil and thus on the person lying on the upper side of the foil. This also opens up the possibility of adjustable immersion end point mounting. In this example too, the region below the sliding foil is heated and kept warm by heating the fluid located in the space below the foil, in particular water, in which the body is immersed with the interposition of the foil. However, warm air heating is also conceivable. In all of the cases, it is ensured that the temperature profile of this medium below the foil is controllable and/or variable, wherein all of the parameters relevant thereto can preferably be adjusted and varied jointly or individually in order to achieve an optimum interplay of the individual measures.

A guest who is located on the foil can therefore use his own body movement to bring his body into a slipping and sliding movement on the foil. This effect can optionally be further intensified by moving, for example via the roll-on and pulling means 29 and/or by means of the diversion rolls 11, for example the sliding foil 10 in all cases by a short distance in one direction and then back again in the other, as a result of which the body is additionally encouraged to slide. This pivoting can ultimately be carried out counter to the force of the spring force storage means 21, for example as a result of (preferably motor-driven) rotation in the same direction caused by the diversion rolls 11. Alternatively or additionally, use may however also be made, for moving the sliding foil 10 back and forth, of the aforementioned roll-on and pulling device 29 which in the exemplary embodiment shown is provided below the carrying device 1, i.e. below the base 7 of the carrying device 1 or of the basic tub element 1′, and comprises two wind-up rolls 31 which can be driven in opposing directions, for example via a motor. These wind-up rolls actuate a foil or belt-like, rope-like or chain-like pulling means 33 which can be diverted about diversion rolls 30 and in the exemplary embodiment shown acts on the rail-like tensioning frame 19′ at the two opposing longitudinal sides 3 of the tub 1′.

In addition, this controlled action can be provided, in order to intensify the sliding effect, by an additionally provided means 151 for tilting or swiveling the device 1 as a whole, i.e. in the exemplary embodiment shown the basic tub element 1′. This is indicated schematically in FIG. 2 as a result of the fact that a swivel or tilt axis 151 is additionally drawn on the underside of the tub in the case of the support and stand means 153 represented therein. This allows, for example, swiveling to be brought about in the direction indicated by the arrow 155. Alternatively and additionally, a further swivel or tilt axis extending transversely to the device can also be formed thereon to allow pivoting of the system as a whole and thus of the foil about an axis extending transversely to the longitudinal direction. If, for example, the device is moved via a longitudinal and a transverse axis simultaneously, if appropriate also by means of a superimposed oscillation and if appropriate even for a period of oscillation of differing duration, this initiates a spatial swiveling movement of the foil, thus allowing a person located thereon more easily to move his body in a sliding manner in relation to the sliding foil carrying him.

Conversely, a means can also be provided, for example using rolls which allow the device as a whole to be adjusted back and forth on the rolls, for example, by a bath attendant or in a motor-driven manner in the sense of a translatory movement in order to assist and to intensify the sliding movement on the foil 10. Equally, the tub could also possibly carry out a rotational movement about a vertical or otherwise inclined axis in order to intensify the sliding movement. It would also be sufficient if merely the diversion means 11 or the diversion rolls 11′ could carry out a corresponding rotational pivoting or tilting or translatory movement relative to the device or to the basic tub element 1′ in order to achieve the same effects. In all cases, this gives rise to a relative movement associated with a shift in the center of gravity of the person located on the foil.

Finally, FIG. 4 also shows that, for example when a guest is located on the foil for treatment, it is possible to fold over from the outside, beyond the edge region 9 of the device and in particular beyond the diversion means 11, holding and gripping means 47 which make it easier for the bather to move his body in a sliding manner on the recessed sliding foil 10 and in order, on the other hand, to give him a greater sense of safety. FIG. 4 is in this case a plan view and FIG. 5 a side view of a holding bar 47 which, adjacent to the lateral diversion rolls 11′, extends in each case over almost the entire length of the device and can be swiveled inward via end-side swiveling levers 47′ from a disengagement position located outside the device to beyond the edge region of the device and can be locked in this position.

An effect heightening the sense of personal well-being can also be achieved as a result of the fact that not only lateral support wedges 49 providing support but rather, for example, also a padded or soft inner part 50 can be provided between the guest's legs for confining an uncontrolled sliding movement. These parts can, for example, likewise be swiveled from the outside inward by means of swivel shafts, so they are at least to a certain extent positionable and their position is not freely variable. Thus, the inner wedge 50 shown in FIGS. 4 and 5 can, for example, be swiveled about a shaft 48′ serving as a foot rest or support 48 from an outwardly swiveled position until it rests on the sliding foil. On the one hand, this allows the body to be stabilized somewhat. On the other hand, outward swiveling of this padded inner part allows the bather to turn from lying on his back into the face-down position and vice versa. Subsequently, this wedge can be swiveled back onto the sliding foil 10.

In addition, supplementary carrying grips 47′ can be configured on this inner part, but also on the lateral support wedge 49, as is also indicated merely schematically in the side view according to FIG. 5.

Finally, FIG. 6 not only shows the inner part 50 which is configured as a knee wedge, thus allowing the guest's knee to be raised somewhat and his legs to be angled, it also shows the use of a back wedge 150. The use of the above-mentioned underlaid inserts 50, 150 allows, for example, the shoulder region and the back and to some extent also the leg and knee regions to be purposefully raised to a somewhat greater extent, so the main weight in the pelvis region now rests on the sliding foil. At this point, which is now the deepest, of the sliding foil additional medium can be introduced, thus producing at least to some extent a limb bath if this is desired.

Alternatively, the aforementioned inner parts 50 can also be rigidly connected to the sliding foil in order at least to some extent to limit the slidable free space at this location. Alternatively, the corresponding insertion parts 50 can also be fixed in a different manner without having to be rigidly connected to the sliding foil 10.

Merely FIGS. 4 and 5 additionally indicate that the head of a guest carrying out the treatment is located preferably outside the carrying device, i.e. the basic tub element 1′, for example can be inserted in a preferably insulating head bowl 51 in order to prevent undesirable movement of the head. The body is also stabilized in this way. In any case, this prevents the body from sliding out of the region of the foil in an uncontrolled manner. If the head and neck support is however fixed loosely to the bath guest's neck, all sliding movements can then be carried out. The head and neck remain padded while being at the same time protected from undesirable heating. Provided in this case is preferably just a support means 3 which is referred to generally as a head rest and is merely indicated in FIGS. 1 and 4.

As may be seen from the diagram, the foil extends to a sufficient degree in the longitudinal direction of the device or tub. In particular if no aqueous fluid is introduced on the underside of the foil, there being just air, heated air or water vapor instead, the dimensions of the foil in the longitudinal direction thereof must merely be such that the foil is able to receive all of the guest's body. Preferably, however, end-side extended portions are provided, although they do not have to be provided. A corresponding head rest can then be provided, at least at an end face of the device. The head rest can however also be arranged within the device or tub with corresponding thermal insulation. Preferably, the head rest is likewise resilient and/or compliant, so no uncomfortable rim is produced at this location when the guest lies down on the foil.

The sliding foil 10 may have become stretched at specific points, in particular as a result of repeated use. This could cause the foil now to sag somewhat more markedly in the horizontal starting position thereof, as a result of which lubricants would on application flow together into the center. This could be prevented by, for example, configuring the foil, as is indicated in the cross-sectional view according to FIG. 7, in a double-walled manner as a hollow body system, the upper cover foil 10a and the lower cover foil 10b being welded to the edges 17 so as to form a foil cavity 161. Also provided is an inlet and/or outlet opening via which a medium, which as a result of an upward arch compensates for the sagging of the foil, can be pumped in. If the upper foil 10a used is then a foil having special sliding properties and a soft grip and a lower foil lob, for example comprising fabric-reinforced materials, which is then intended primarily to accommodate all of the carrying force, the upper sliding foil 10a is thus supported.

Moreover, suitable gaseous or preferably free-flowing medium, including thick paste-like or gel-like medium, could be pumped as appropriate through the aforementioned filling opening into the cavity 161 to a degree such that even when a bath guest lies down on the device, the upper surface, i.e. the sliding foil 10a, is and remains oriented more or less horizontally or has only a slightly concave depression, wherein the degree of arching can be set by way of the medium which is added by pumping and rests on the lower carrying foil 10b.

A double foil of this type comprising at least two foil layers, which can be welded, bonded to one another or formed so as to form the aforementioned foil cavity 161, allows the formation of a foil upper layer having optimum sliding properties and a lower foil carrying layer which has sufficient dimensions by comparison or is provided with fabric materials.

Therefore, there can also be provided a preferably fabric-reinforced foil which accommodates sufficient carrying forces and is equipped at the surface thereof with its particularly effective sliding layer as the upper side 10a of the foil. In this case, no cavity would therefore be provided.

However, as FIG. 5 shows, there is preferably also provided a third foil 1c or foil layer which is provided below the actual fabric-reinforced carrying foil 10b and droops down deeper into the device from the lateral diversion rolls. This third foil or foil layer 1c is also tightly connected, for example welded, to the other foil layers, preferably at the edge region 17, 17′, thus producing a further closed cavity 163. This third foil 10c is preferably likewise again fabric-reinforced so, in the event of the upper fabric-reinforced foil or foil layer 10b breaking, at least this foil which hangs down lower can still intercept a bather.

The lower cavity 163 is provided at least with a supply opening, preferably a feed opening or a discharge opening 165, 167, where for example heatable air can be supplied. This allows the system as a whole to be heated or brought to a specific temperature, as the temperature then rises, above the upper foils 10b and 10a and the compensating medium which may be located therebetween, up to the bath guest. If an adjustable restrictor valve is provided in the return or if the outflow opening is made smaller, a certain excess pressure is ensured at all times in the lower heating chamber 163.

Setting the pressure accordingly also allows the horizontal position of the top sliding layer 10a to be set accordingly. For the purposes of cleaning the device as a whole, the excess pressure can even be increased in such a way that the top contact surface 10′ of the sliding foil 10a even bulges in an at least slightly convex manner, so the cleaning agent and the cleaning liquid can run off laterally via the foil.

Preferably not only gaseous or free-flowing but rather also paste-like or thick paste-like or gel-like medium can also be introduced in the upper chamber 161, thus allowing beneficial effects to be achieved under certain circumstances. Especially if use is made of a third lower foil 10c, of which the cavity 163 located thereabove can be loaded with pressure and filled to differing degrees by adding a liquid or preferably gaseous medium by pumping (in order to reduce the weight), provision may also be made for the cavity 161 between the top sliding foil 10a and the first carrying foil 10b located therebelow to be permanently tightly closed. The filling of the lower space 163 with gas likewise allows, again, the degree of sagging or the horizontal orientation of the top sliding foil 10a to be set. For the purposes of cleaning, the cavity 163 can be inflated in such a way that a convex curvature is set at the upper foil 10a, so cleaning water runs off laterally via the foil.

The third, i.e. bottom, foil 10c used and the chamber 163 which is thereby formed and serves as a heating chamber ultimately allow a basic tube element 1′ to be dispensed with entirely. Indeed, all that is required is a simple carrying device 1 which can be set up, for example, on four feet 1a, there being provided in this case on the upper, preferably peripheral frame the at least opposing lateral contact edges 9 and/or the diversion rolls 11′ which are positioned at this location, in order to carry and to support the foil 10.

1. A device for carrying out a treatment, namely using lubricant in the form of liquid, free-flowing, paste-like, creamy, sliding and/or rolling additives, having the following features:
comprising a frame-like carrying means, preferably in the form of a basic tub element,
a foil is held on the carrying device,
the foil is held and/or supported at least in certain portions and/or on opposing sides at a peripheral edge of the carrying device,
the foil sags in relation to a horizontal plane during the treatment, comprising the following further features:
the foil is located for carrying out the treatment in a position or can be brought for carrying out the treatment into a position in which the lateral portions of the foil, which lead to the lateral edge support thereof, ascend in relation to a horizontal plane at an angle smaller than 45°,
the device is configured in such a way that the foil forms a contact surface which is configured in a bowl, plate or wok-like or concave manner, and
the foil is, as a sliding foil, of such configuration and composition that its upper side is slidable at least with additional use of treatment medium and/or lubricant.

2. The device as claimed in claim 1, wherein when a person lies down on the device the foil portions leading laterally from the person to the edge portions ascend at an angle smaller than 40°, in particular smaller than 35°, 30° and/or 25°.

3. The device as claimed in claim 1, wherein the foil is held at least in certain portions at least two opposing sides of the carrying device.

4-6. (canceled)

7. The device as claimed in claim 1, wherein the foil comprises a fabric reinforcement or is underlaid with a fabric reinforcement.

8. The device as claimed in claim 1, wherein the foil is provided with a highly slidable contact surface.

9. The device as claimed in claim 1, wherein the foil has at least two layers and comprises a highly slidable top foil and a carrying foil which is located therebelow and preferably has a fabric reinforcement.

10-16. (canceled)

17. The device as claimed in claim 1, further including a downwardly sagging foil arranged below the foil and wherein the downwardly sagging foil likewise performs the carrying function, preferably using a fabric reinforcement.

18. The device as claimed in claim 1, wherein the foil preferably comprising the individual foils is held by a carrying device provided with a stand or foot means.

19. The device as claimed in claim 1, wherein a means is provided for raising and lowering the foil.

20. The device as claimed in claim 1, wherein the foil assumes in cross section a slight bowl or wok-like shape when a person lies down on the device.

21. The device as claimed in claim 1, wherein inserts which interact with the underside of the foil are present in the space below the foil.

22. The device as claimed in claim 21, wherein the inserts are made of resilient and/or deformable materials.

23. The device as claimed in claim 22, wherein the at least one insert is made of foam rubber or foam or a comparable material, so a person located on the foil lies from the upper side onto the insert thus formed, preferably at least slightly compressing this insert.

24. The device as claimed in claim 21, wherein the insert is made of preferably floatable granular material.

25. The device as claimed in claim 1, wherein the carrying device comprises a basic tub element and wherein liquid and/or gaseous medium can flow through the space between the underside of the foil and the interior of the tub, preferably in the form of a circulation means using at least one feed tube and at least one discharge tube.

26. The device as claimed in claim 1, wherein at least two lateral holding and/or gripping means can be brought, pivoted and/or transferred from the outside at least into the edge region of the foil and can preferably be fixed in this position.

27. The device as claimed in claim 1, wherein there are provided on the foil, separated therefrom, lateral, preferably padded support means and/or a preferably resilient, soft and/or padded inner part and/or knee rest part.

28. The device as claimed in claim 17, wherein the at least one support means and/or an inner or knee rest part can be pivoted outward from a use position located on the sliding foil into a disengagement position.

29. The device as claimed in claim 27, wherein the inner and/or knee rest part Scan be pivoted about a foot support.

30. The device as claimed in claim 27, wherein the at least one support means and/or the inner and/or knee rest part are provided with gripping means.

31. The device as claimed in claim 1, wherein there is provided a means which sets in motion manually and/or in a motor-driven manner at least the means for holding and diverting the foil and preferably the entire carrying device or the basic tub element.

32. The device as claimed in claim 31, wherein the means allows a translatory movement, a tilting or swiveling movement and/or at least a rotational movement at least of the sliding foil, i.e. preferably including the diversion means carrying said foil.

33. The device as claimed in claim 32, wherein the corresponding means is provided below the carrying device or the basic tub element.

34. (canceled)

35. The device as claimed in claim 1, wherein a chamber formed below the top foil can be inflated in such a way that the top foil assumes a convex curvature to allow cleaning water to run off laterally.

36. The device as claimed in claim 1, wherein a fan or the like, by means of which water can be distributed for cleaning the lying surface and optionally a bath guests located on the lying surface, is further provided above the device.
